(19) United States
(12) Patent Application Publication
(10) Pub. No.: US 2013/0211418 A1
(43) Pub. Date: Aug. 15, 2013

(54) APPARATUS AND METHOD FOR TACTILE FEEDBACK

(71) Applicants: Soo Chul Lim, Seoul (KR); Hyung Kew Lee, Gunpo-si (KR); Joon Ah Park, Seoul (KR); Bho Ram Lee, Seongnam-si (KR); Hyun Jeong Lee, Hwaseong-si (KR); Seung Ju Han, Seoul (KR)

(72) Inventors: Soo Chul Lim, Seoul (KR); Hyung Kew Lee, Gunpo-si (KR); Joon Ah Park, Seoul (KR); Bho Ram Lee, Seongnam-si (KR); Hyun Jeong Lee, Hwaseong-si (KR); Seung Ju Han, Seoul (KR)

(73) Assignee: Samsung Electronics Ltd., Co., Suwon (KR)

(21) Appl. No.: 13/494,576

(22) Filed: Jun. 12, 2012

(30) Foreign Application Priority Data
Feb. 10, 2012 (KR) 10-2012-0013766

(51) Int. Cl. A61B 19/00; G08B 6/00

(52) U.S. Cl. 606/130; 340/407.2

(57) ABSTRACT

An apparatus and method for a tactile feedback are provided. The tactile feedback apparatus may include a position measurement unit to measure a position of a mechanical link, and a tactile feedback unit to transmit a tactile sensation based on at least one of a position of the mechanical link and a rotation angle of the mechanical link.

### CROSS-REFERENCE TO RELATED APPLICATIONS

This application claims the priority benefit of Korean Patent Application No. 10-2012-0013766, filed on Feb. 10, 2012, in the Korean Intellectual Property Office, the disclosure of which is incorporated herein by reference.

### BACKGROUND

1. Field

One or more example embodiments of the following description relate to an apparatus and method for providing a tactile feedback by attaching a device, that transmits a texture to a human finger, to a device that measures a spatial position, and more particularly, to an apparatus and method for transmitting feedback information including a force vector, a texture, and the like, to a tactile organ of a human, such as a finger, by expressing the feedback information using a physical movement.

2. Description of the Related Art

Generally, a technology for transmitting a force or a tactile sensation is referred to as tactile feedback. Here, as a force applied to a robot is transmitted to a human finger in greater detail, the robot may be manipulated in more detail, for example, with greater precision. More specifically, “tactile feedback” refers to generation and transmission of an artificial sense to similarly reproduce intuitional manipulation of an object in a virtual space or remote manipulation of a robot.

For example, when a human operator manipulates a conventional surgical robot, since force transmission is performed unidirectionally, a tension, a load, and other physical quantities representing a force applied to the surgical robot may not be fed back in the form of a tactile sense to the human operator.

That is, a conventional kinesthetic device may not transmit, to a human operator, a texture and the like generated as a robot touches a particular object located nearby, besides a force applied to the robot. Therefore, operation stability may be reduced.

Accordingly, there is a demand for a new technology capable of feeding back a texture, a force, and the like generated as a robot touches a particular object located proximate to a human operator in the form of a tactile sense, thereby increasing stability.

### SUMMARY

The foregoing and/or other aspects are achieved by providing a tactile feedback apparatus including a position measurement unit to measure a position of a mechanical link; and a tactile feedback unit to transmit a tactile sensation based on at least one of a position of the mechanical link and a rotation angle of the mechanical link.

The tactile feedback unit may be attached to at least one mechanical link included in the position measurement unit to transmit the tactile sensation.

The tactile feedback apparatus may further include an operation control unit attached to the mechanical link and configured to perform a forceps operation.

The tactile feedback unit may be attached to an outer surface of the forceps included in the operation control unit to transmit the tactile sensation.

The tactile feedback unit may transmit the tactile sensation by moving at least one pin in at least one of a horizontal direction, a forward and backward direction, and a vertical direction, based on the position of the mechanical link and the rotation angle of the mechanical link.

The tactile feedback unit may have three degrees of freedom for moving the at least one pin in at least one of the horizontal direction, the forward and backward direction, and the vertical direction, using a piezo actuator, an ultrasonic actuator, or an electro active polymer.

The tactile feedback unit may transmit at least one of a force, a movement, a texture, and a surface shape applied to an object to human skin by moving the at least one pin.

The tactile feedback may transmit the tactile sensation by adjusting a pneumatic pressure of a balloon according to the position of the mechanical link and the rotation angle of the mechanical link.

The tactile feedback unit may transmit oscillation or vibration to the human skin based on the position of the mechanical link and the rotation angle of the mechanical link.

The tactile feedback unit may transmit a force generated by a forceps operation of a remote controlled surgical robot to human skin.

The foregoing and/or other aspects are achieved by providing a tactile feedback method including measuring a position of a mechanical link; and transmitting a tactile sensation to human skin based on at least one of the position of the mechanical link and a rotation angle of the mechanical link.

The transmitting may include transmitting the tactile sensation using a tactile feedback unit attached to at least one mechanical link included in a position measurement unit.

The tactile feedback method may further include performing a forceps operation using an operation control unit attached to the mechanical link.

The transmitting may include transmitting the tactile sensation by a tactile feedback unit attached to an outer surface of forceps included in the operation control unit.

The transmitting may include transmitting the tactile sensation by moving at least one pin in at least one of a horizontal direction, a forward and backward direction, and a vertical direction, based on the position of the mechanical link and the rotation angle of the mechanical link.

The transmitting may include moving the at least one pin in at least one of the horizontal direction, the forward and backward direction, and the vertical direction, using a piezo actuator, an ultrasonic actuator, or an electro active polymer.

The transmitting may include transmitting at least one of a force, a movement, a texture, and a surface shape applied to an object to the human skin by moving the at least one pin.

The transmitting may include transmitting the tactile sensation by adjusting a pneumatic pressure of a balloon according to the position of the mechanical link and the rotation angle of the mechanical link.

The transmitting may include transmitting oscillation or vibration to human skin based on the position of the mechanical link and the rotation angle of the mechanical link.

The transmitting may include transmitting at least one of kinesthetic feedback and tactile feedback to human skin based on the position of the mechanical link and the rotation angle of the mechanical link.

The foregoing and/or other aspects are achieved by providing a tactile feedback apparatus including a position measurement unit to measure a position of a mechanical link and a tactile feedback unit to transmit a tactile sensation by moving at least one pin based on a position of the mechanical link.

The foregoing and/or other aspects are achieved by providing a system for performing remotely-controlled surgery. The system includes a surgical robot comprising a surgical tool, the surgical robot to measure a force generated by the surgical tool and a tactile feedback apparatus to transmit a tactile sensation to a hand of a surgeon based on the force generated by the surgical tool of the surgical robot.

The tactile sensation transmitted by the tactile feedback unit includes one or more of a force, a movement, a texture, and a surface shape, corresponding to the force generated by the surgical tool.

Additional aspects, features, and/or advantages of example embodiments will be set forth in part in the description which follows and, in part, will be apparent from the description, or may be learned by practice of the disclosure.

### BRIEF DESCRIPTION OF THE DRAWINGS

These and/or other aspects and advantages will become apparent and more readily appreciated from the following description of the example embodiments, taken in conjunction with the accompanying drawings of which:

FIG. 1 illustrates a tactile feedback apparatus including a tactile feedback unit, according to one or more example embodiments;

FIG. 2 illustrates a sectional view of a tactile feedback unit employing an actuator, which is included in the tactile feedback apparatus shown in FIG. 1;

FIG. 3 illustrates a sectional view of a tactile feedback unit using pneumatic pressure, which is included in the tactile feedback apparatus shown in FIG. 1;

FIG. 4 illustrates a sectional view of a tactile feedback unit using oscillation, which is included in the tactile feedback apparatus shown in FIG. 1;

FIG. 5 illustrates of a tactile feedback apparatus including a tactile feedback unit attached to an operation control unit, according to one or more example embodiments;

FIG. 6 illustrates a perspective view of the operation control unit shown in FIG. 5;

FIG. 7 illustrates an entire system of a tactile feedback apparatus that transmits a tactile sensation to human skin using a surgical robot, according to one or more example embodiments;

FIG. 8 illustrates an entire system of a tactile feedback apparatus that transmits a tactile sensation to human skin using a surgical robot and a camera, according to one or more example embodiments; and

FIG. 9 illustrates an operation of transmitting a tactile sensation from the tactile feedback apparatus shown in FIG. 1 to human skin.

### DETAILED DESCRIPTION

Reference will now be made in detail to example embodiments, examples of which are illustrated in the accompanying drawings, wherein like reference numerals refer to the like elements throughout. One or more example embodiments are described below to explain the present disclosure by referring to the figures.

According to one or more example embodiments, feedback information including a force vector and a texture caused by manipulation of a robot located in a remote position or an object located in a virtual space may be transmitted in detail to a finger or to the skin of a human. Thus, tactile feedback may be provided.

In addition, since a tactile sensation is transmitted to the human skin, operational efficiency and safety using the robot or the object may be increased.

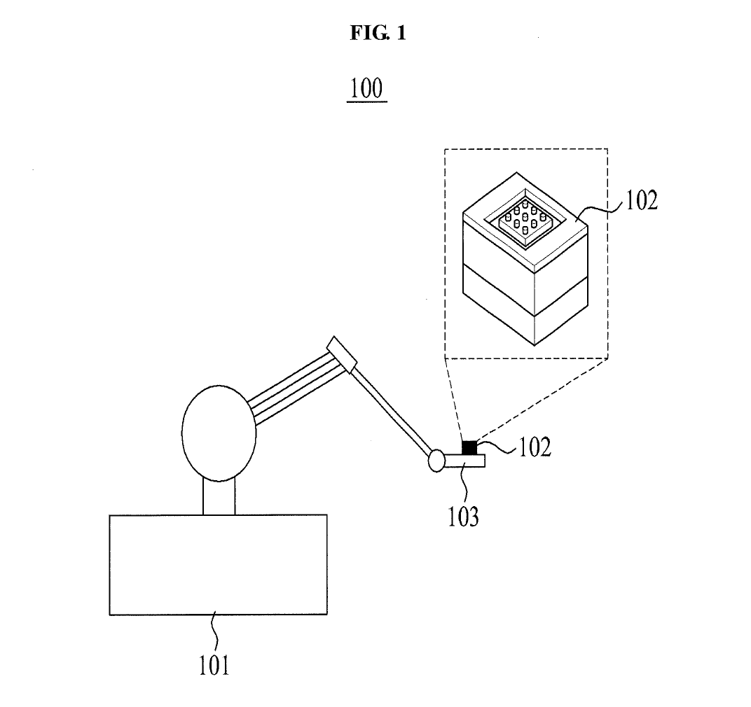

FIG. 1 is a diagram of a tactile feedback apparatus 100 including a tactile feedback unit 102, according to example embodiments.

According to FIG. 1, the tactile feedback apparatus 100 may include, for example, a position measurement unit 101 and the tactile feedback unit 102.

The position measurement unit 101 may include at least one mechanical link and may be used to measure a position of the at least one mechanical link. For example, the position measurement unit 101 may measure a position of a handle unit among the at least one mechanical link. Here, the mechanical link refers to a unit including a motor and an encoder. The position measurement unit 101 may measure a rotation angle and a spatial position of the at least one mechanical link using the motor and the encoder. For example, the position measurement unit 101 may measure a 3-dimensional (3D) position of the at least one mechanical link.

The tactile feedback unit 102 may transmit a tactile sensation to human skin based on at least one of the position of the at least one mechanical link and the rotation angle of the at least one mechanical link. Here, as an example, the tactile feedback unit 102 may transmit the tactile sensation based on the position and the rotation angle of the handle unit of the at least one mechanical link.

For example, when an object located in a virtual space or in a remote location collides and makes contact with an object or human located nearby, the tactile feedback unit 102 may transmit a tactile sensation such as a force, a movement, a texture, a surface shape, and the like applied to the object at the time contact is made, to human skin in the form of a tactile sense. Thus, by transmitting the tactile sensation the tactile feedback unit 102 may attempt to simulate the sensation caused by contact between the object located in the virtual space and the object or the human located nearby. The object herein may include a robot disposed at the remote position and various other objects present in the virtual space.

The tactile feedback unit 102 may be attached to at least one mechanical link included in the position measurement unit 101. For example, the tactile feedback unit 102 may be attached to a last mechanical link 103 among the at least one mechanical link included in the position measurement unit 101. Here, the last mechanical link 103 may be a handle portion for a human user to grab to control operation of the object such as a robot.

In addition, the tactile feedback unit 102 may transmit the tactile sensation by moving at least one pin in at least one of a horizontal direction, a forward and backward direction, and a vertical direction based on the position and the rotation angle of the mechanical link. Here, the tactile feedback unit 102 may move the at least one pin in at least one of the horizontal direction, the forward and backward direction, and the vertical direction, using a piezo actuator, an ultrasonic actuator, or an electro active polymer. Also, the tactile feedback unit 102 may transmit the tactile sensation by adjusting a pneumatic pressure of a balloon, instead of using the actuator or the electro active polymer.

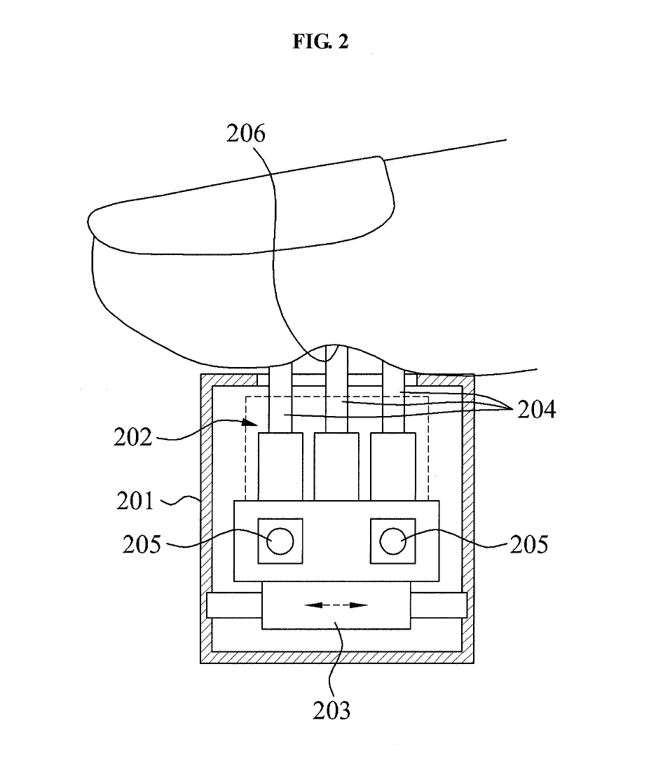

FIG. 2 illustrates a structure of a tactile feedback unit 200 employing an actuator, which may be included in the tactile feedback apparatus 100 shown in FIG. 1.

Referring to FIG. 2, the tactile feedback unit 200 may include, for example, a fixing portion 201, a movable portion 202, a first driving portion 203, a second driving portion 204, and a third driving portion 205. The first 203, second 204, and third driving portion 205 collectively may provide for movement in a first, second, and third dimension such as a horizontal direction, a forward and backward direction, and a vertical direction.

The fixing portion 202 refers to a body such as an enclosure surrounding the movable portion and the first driving portion 203. The fixing portion 202 may define a movable range of the movable portion 202.

The movable portion 202 may transmit a tactile sensation to human skin by moving within the fixing portion 202. For example, the movable portion 202 may move in at least one of a horizontal direction (x-axis direction), a vertical direction (y-axis direct ion), and a forward and backward direction (z-axis direction). A contact surface of the movable portion 202 may transmit the tactile sensation through contact with human skin such as an inner surface 206 of a finger.

The first driving portion 203 to the third driving portion 205 may control the movable portion 202 to move in at least one direction in accordance with an input signal. The input signal may be a feedback signal including a load applied to an object while the object is being controlled remotely. For example, the input signal may include a signal corresponding to a force, a movement, a texture, and a shape surface which are applied to the object.

The first driving portion 203, the second driving portion 204, and the third driving portion 205 may simultaneously control a movement of the movable portion 202 and a pin array, in the horizontal direction, the vertical direction, and the forward and backward direction, respectively. Accordingly, a more detailed and realistic tactile stimulus may be generated and transmitted to the human skin.

First, the first driving portion 203 may control a horizontal movement of the movable portion 202 based on the input signal. In this instance, the first driving portion 203 may use a piezo motor to move the movable portion 202 horizontally to the left or to the right or in both directions.

For example, when the object is moved to the left or the right, the input signal may include a motion vector representing a speed and direction of the movement of the object. Therefore, the first driving portion 203 may move the movable portion 202 to the left or the right according to the movement speed based on the input signal. As a result, the contact surface of the movable portion 202 and a contact surface of at least one pin constituting the pin array may transmit the movement applied to the object, to human skin through contact with the inner surface 206 of the finger.

The second driving portion 204 may include the pin array including at least one pin and may be inserted in the movable portion 202. Each of the at least one pin constituting the pin array may move independently. The second driving portion 204 is disposed at an upper end of the third driving portion 205 and configured to control movements of the at least one pin up and down, that is, in the vertical direction based on the input signal. The vertical direction may be a direction that is orthogonal to the inner surface 206 of the finger.

For example, when the object pushes an uneven surface hard, such as with great force, the input signal may include a force vector representing a texture of the uneven surface and a pushing force. Therefore, the second driving portion 204 may move the at least one pin constituting the pin array to respectively different heights based on the input signal transmitted from the object. Accordingly, the contact surface of the at least one pin may transmit the texture of the uneven surface, the force, and the surface shape which are applied to the object to the human skin through contact with the inner surface 206 of the finger at the different respective heights.

According to another example, when the object moves along a surface inclined from the upper left to the lower right at a speed “a”, the first driving portion 203 may move the movable portion 202 to the right based on the input signal while the second driving portion 204 controls the height of the at least one pin based on the input signal. For example, the height of the at least one pin of the pin array may be reduced toward the right. In this case, the first driving portion 203 and the second driving portion 204 may move the movable portion 202 and the pin array simultaneously. For example, when the movable portion 202 is moved to the right at the speed a, the contact surface of the pin array may be moved to the right at the speed “a” and reduced in height corresponding to the movement of the movable portion 202, thereby contacting the inner surface 206 of the finger. Accordingly, the contact surface may transmit the movements performed in the vertical direction and the horizontal direction, the force, the inclined surface, and the like applied to the object to the human skin simultaneously.

The third driving portion 205 may control the movement of the movable portion 202 in the forward and the backward direction based on the input signal. Here, the third driving portion 205 may be disposed at a lower end of the movable portion 202 and an upper end of the first driving portion 203. For example, in an embodiment, the third driving portion 205 may be disposed between the movable portion 202 and the first driving portion 203. The third driving portion 205 may move the movable portion 202 using at least one piezo motor.

For example, when the object is moved in a diagonal direction, the first driving portion 203 may move the movable portion 202 in the horizontal direction based on the input signal while the third driving portion 205 moves the movable portion 202 in the forward and the backward direction based on the input signal. In this instance, as the first driving portion 203 and the third driving portion 205 move the movable portion 202 in the horizontal direction and the forward and the backward direction simultaneously, the diagonal movement applied to the object may be transmitted to the human skin.

As aforementioned, the tactile feedback apparatus 100 shown in FIG. 1 may transmit not only a kinesthetic feedback but also a tactile feedback using the tactile feedback unit attached to the position measurement unit. That is, the tactile feedback apparatus 100 may transmit, to the human skin, not only the force and the movement applied to the object but also the texture and the surface shape as the tactile sensation.

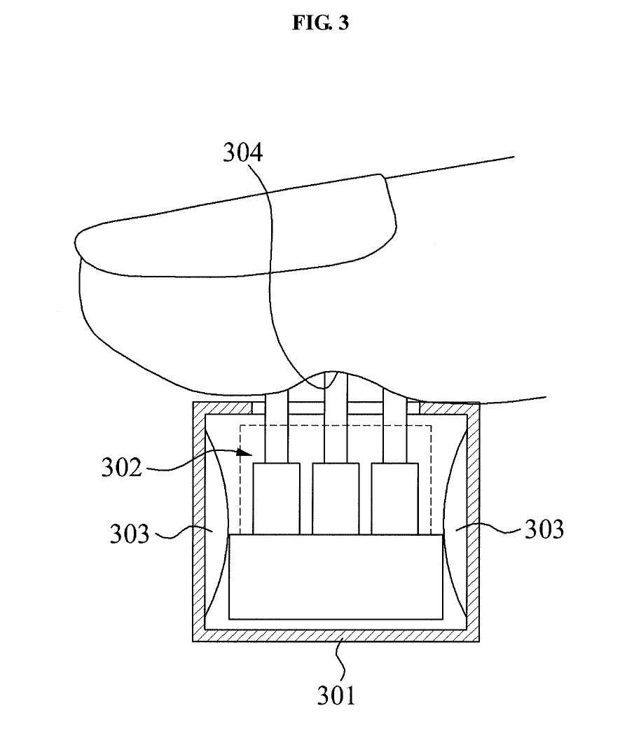

FIG. 3 illustrates a structure of a tactile feedback unit 300 using a pneumatic pressure, which is included in the tactile feedback apparatus 100 shown in FIG. 1.

According to FIG. 3, the tactile feedback unit 300 may include, for example, a fixing portion 301, a movable portion 302, and a driving portion 303. The tactile feedback unit shown in FIG. 3 employs a balloon instead of the piezo motor used in the tactile feedback unit of FIG. 2 to control the movement of the movable portion in the horizontal direction and the forward and backward direction. Therefore, the tactile feedback unit 300 may transmit the tactile sensation to human skin by controlling the pneumatic pressure of the balloon in accordance with an input signal generated based on the position and the rotation angle of the at least one mechanical link.

The driving portion 303 may move the movable portion 302 in a horizontal direction and a forward and backward direction by controlling the pneumatic pressure of the balloon based on the input signal. Here, the driving portion 303 may be disposed to contact an inner surface of the fixing portion 301.

For example, air may be injected through an air inlet of the balloon using an air injection device based on the input signal. Therefore, the balloon may gradually swell, thereby applying a force to the movable portion 302. Consequently, the movable portion 302 may be moved in the force transmission direction. The movement may generate a tactile stimulus through contact surfaces of the movable portion 302 and the pin array contacting an inner surface 304 of a finger.

Thus, the driving portion 303 may move the movable portion 302 in the horizontal direction and the forward and backward direction by transmitting the force to the movable portion 302 by controlling the pneumatic pressure of balloons disposed at each of the four sides, respectively.

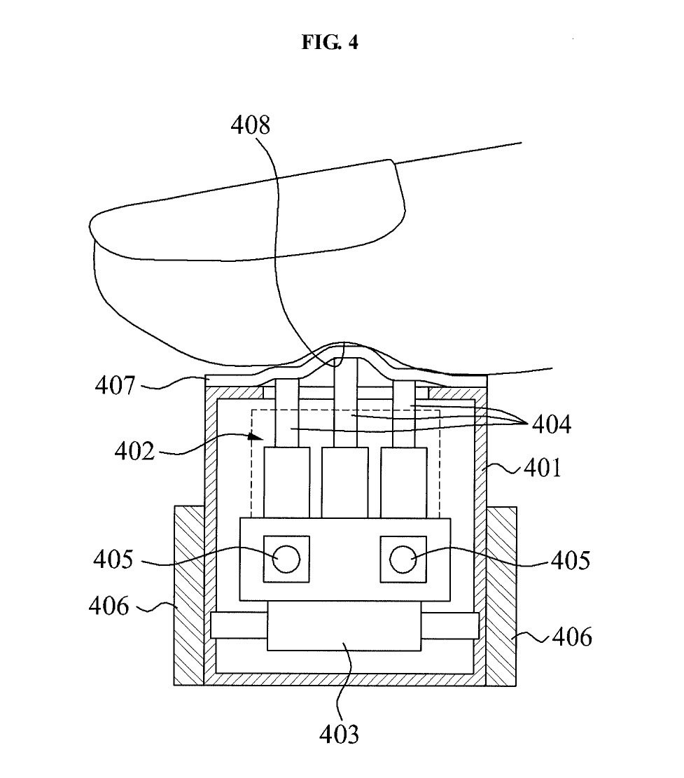

FIG. 4 illustrates a structure of a tactile feedback 400 using oscillation or vibration, which is included in the tactile feedback apparatus 100 shown in FIG. 1.

According to FIG. 4, the tactile feedback unit 400 may include, for example, a fixing portion 401, a movable portion 402, a first driving portion 403, a second driving portion 404, a third driving portion 405, and an oscillation unit 406. Here, the tactile feedback unit 400 may further include an intermediate medium 407.

Since the fixing portion 401, the movable portion 402, the first driving portion 403, the second driving portion 404, and the third driving portion 405 operate in substantially the same manner as the fixing portion 201, the movable portion 202, the first driving portion 203, the second driving portion 204, and the third driving portion 205, a repeated description will be omitted for conciseness.

The oscillation portion 406 may transmit oscillations or vibrations to human skin based on the position and the rotation angle of the at least one mechanical link. That is, the oscillation portion 406 may transmit oscillations or vibrations to the movable portion 402 in accordance with the input signal generated based on the position and the rotation angle of the at least one mechanical link. The oscillation portion 406 may be disposed in contact with an outer surface of the fixing portion 401. For example, the oscillation portion 406 may be in contact with at least one of four outer surfaces of the fixing portion 401.

Here, the oscillation portion 406 may generate a high-frequency oscillation using an oscillator, thereby transmitting an oscillating feeling or a vibrating sensation to the inner surface 406 of the finger.

For example, when the object touches a particular surface, that is, when a force vector is too small to generate a tactile sensation to the human skin merely by adjusting the height of the pin array, the oscillation portion 406 may generate a minute oscillation and transmit the minute oscillation to the inner surface 406 of the finger, thereby generating a tactile stimulus with respect to an extremely small force. Furthermore, through the minute oscillation, the oscillation portion 406 may transmit, to human skin, a minor change of the particular surface as well as the texture of the particular surface contacting the object.

Here, the oscillation portion 406 may generate oscillations simultaneously when the first driving portion 402, the second driving portion 403, and the third driving portion 404 control the movement of the movable portion 402 and the pin array in the horizontal, vertical, and forward and backward directions. Accordingly, the oscillation portion 406 may transmit a minor change applied to the object in the horizontal, vertical, and forward and backward directions, to the human skin.

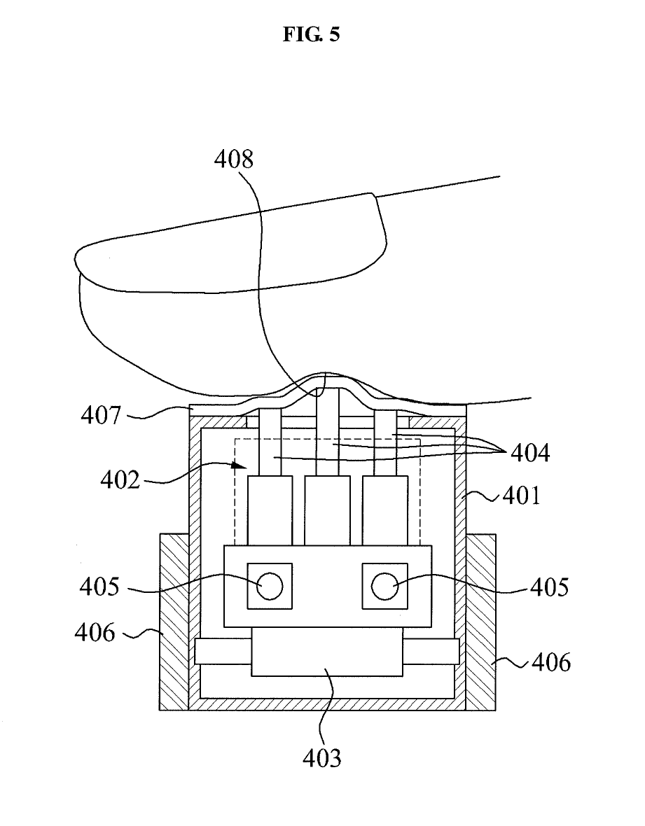

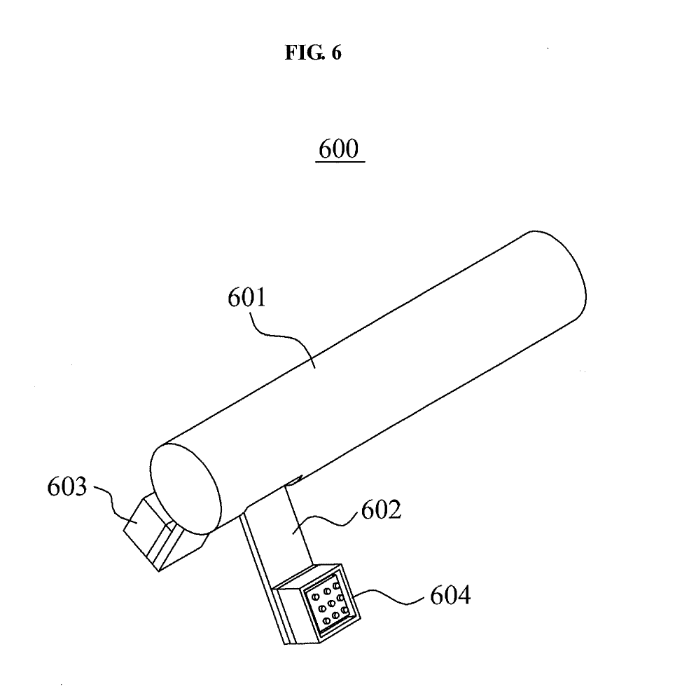

FIG. 5 illustrates a structure of a tactile feedback apparatus 500 including a tactile feedback unit 502 attached to an operation control unit 503, according to example embodiments. FIG. 6 is a perspective view of an operation control unit 600 shown in FIG. 5.

According to FIG. 5, the tactile feedback apparatus 500 may include, for example, a position measurement unit 501, the tactile feedback unit 502, and the operation control unit 503.

Since the position measurement unit 501 and the tactile feedback unit 502 operate in substantially the same manner as the position measurement unit 101 and the tactile feedback unit 102, a detailed description will not be repeated.

The operation control unit 503 may be attached to a last mechanical link 504 among at least one mechanical link included in the position measurement unit 501 to perform an operation such as a forceps operation. For example, when remotely controlling a surgical robot, the operation control unit 503 may perform the forceps operation so that the surgical robot controls a forceps of a surgical tool.

Here, the tactile feedback unit 502 may be attached to an outer surface of the forceps included in the operation control unit 503 to transmit the tactile sensation to human skin. As shown in FIG. 6, a tactile feedback unit 602 may include, for example, a first tactile feedback unit 603 and a second tactile feedback unit 604.

Referring to FIG. 6, the operation control unit 600 may include a handle portion 601 and a forceps 602 for a human user to perform the forceps operation through contact with a thumb and an index finger. Therefore, the first tactile feedback unit 603 may be attached to an upper surface of a portion of the forceps for contacting the index finger. The second tactile feedback unit 604 may be attached to an upper surface of a portion of the forceps for contacting the thumb.

The first tactile feedback unit 603 and the second tactile feedback unit 604 may sense a relative distance between the thumb and the index finger and transmit a forceps angle of the surgical robot to the human skin. For example, the first tactile feedback unit 603 and the second tactile feedback unit 604 may sense the relative distance using an acceleration sensor or a gyro sensor equipped to the handle unit 601. Therefore, the operation control unit 600 may control the forceps operation of the surgical robot by fitting a distance between the first tactile feedback unit 603 and the second tactile feedback unit 604 to the sensed relative distance.

Here, the first tactile feedback unit 603 and the second tactile feedback unit 604 may transmit a force generated by the forceps operation of the surgical robot being controlled by remote, to human skin such as the skin of one or more fingers.

For example, when the surgical tool of the surgical robot touches an organ or bone of a person undergoing surgery or performs a surgical operation such as holding a needle, the first tactile feedback unit 603 and the second tactile feedback unit 604 may transmit a tactile feedback to human skin. That is, the first tactile feedback unit 603 and the second tactile feedback unit 604 may transmit the tactile feedback such as a texture and a surface shape in a direction of the force generated as the forceps touches a human tissue, organ, or bone.

In addition, the first tactile feedback unit 603 and the second tactile feedback unit 604 may transmit the tactile feedback to the human skin using a piezo actuator, an ultrasonic actuator, an electro active polymer, a pneumatic pressure, or an oscillation.

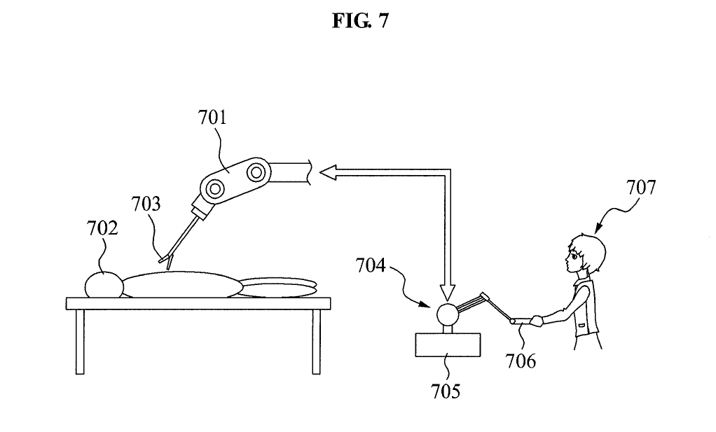

FIG. 7 illustrates an entire system of the tactile feedback apparatus that transmits a tactile sensation to human skin using a surgical robot, according to example embodiments.

According to FIG. 7, a surgical robot 701 that is utilized to perform surgery on a patient 702 may sense whether a surgical tool 703 touches an object located nearby. In addition, the surgical robot 701 may transmit sensed information to the tactile feedback apparatus 704 in a wired or wireless manner.

Therefore, the tactile feedback apparatus 704 may be controlled by remote such that a position of a tip of the surgical tool 703 is changed using the position measurement unit 705. In addition, using a tactile feedback unit 706, the tactile feedback apparatus 704 may transmit a force, a texture, and a surface shape applied as the tip of the surgical tool 703 touches the object, in the form of a tactile sensation to a hand of a surgeon 707. Here, the position measurement unit 705 may be attached to both outer surfaces of a forceps contacting a thumb and an index finger of the doctor 707 to transmit the tactile feedback, thereby increasing safety.

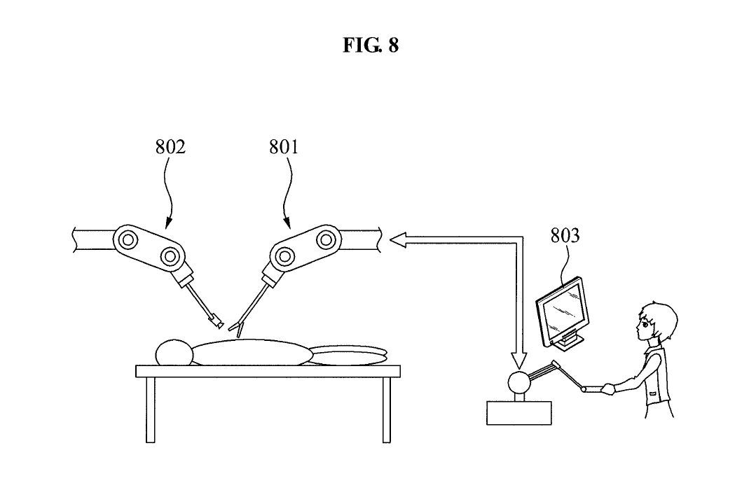

FIG. 8 illustrates an entire system of a tactile feedback apparatus that transmits a tactile sensation to human skin using a surgical robot and a camera, according to example embodiments.

According to FIG. 8, a surgical robot 801 may perform surgery using a surgical tool. A camera-equipped robot 802 may photograph an operation of the surgical robot 801. Also, the camera-equipped robot 802 may transmit the photographed image to a display 803 in a wired or wireless manner. Accordingly, a surgeon 805 may remotely control the surgery being performed by the surgical robot 801, based on the photographed image displayed through the display 803 and a tactile feedback transmitted through a tactile feedback unit.

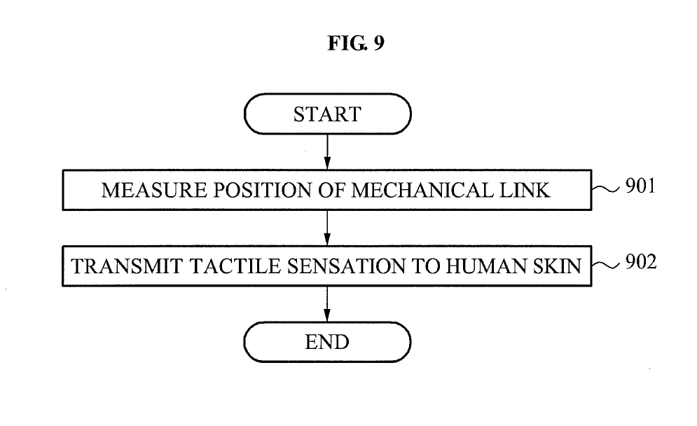

FIG. 9 illustrates an operation of transmitting a tactile sensation from the tactile feedback apparatus 100 shown in FIG. 1 to human skin.

In operation 901, the tactile feedback apparatus may measure a position of at least one mechanical link included in the position measurement unit. For example, the tactile feedback apparatus may measure a position of a handle portion among the at least one mechanical link. The mechanical link may be a unit including a motor and an encoder. Accordingly, the tactile feedback apparatus may measure a rotation angle and a spatial position of the at least one mechanical link using the motor and the encoder.

In operation 902, the tactile feedback apparatus may transmit a tactile sensation to human skin based on at least one of the position and the rotation angle of the at least one mechanical link. In this instance, the tactile feedback apparatus may transmit the tactile sensation to the human skin using the tactile feedback unit attached to the at least one mechanical link included in the position measurement unit. For example, the tactile feedback unit may be attached to a last mechanical link which makes contact with the human skin among the at least one mechanical link included in the position measurement unit.

The tactile feedback apparatus may perform a forceps operation using the operation control unit attached to the at least one mechanical link. As a result, the tactile feedback unit attached to an outer surface of a forceps included in the operation control unit may transmit the tactile sensation to the human skin.

Here, the tactile feedback apparatus may transmit the tactile sensation by moving at least one pin in at least one of a horizontal direction, a forward and backward direction, and a vertical direction, based on the position and the rotation angle of the at least one mechanical link.

For example, the tactile feedback apparatus may use a piezo actuator, an ultrasonic actuator, or an electro active polymer to move the at least one pin in the at least one of the horizontal direction, the forward and backward direction, and the vertical direction. Thus, the tactile feedback apparatus may provide not only a kinesthetic feedback but also a tactile feedback by transmitting a force, a movement, a texture, and a surface shape applied to the object to the human skin by moving the at least one pin.

As another example, the tactile feedback apparatus may transmit the tactile sensation using a pneumatic pressure of a balloon instead of using an actuator. For example, the tactile feedback apparatus may transmit the tactile sensation by adjusting a pneumatic pressure of a balloon according to the position and the rotation angle of the at least one mechanical link.

Alternatively, the tactile feedback apparatus may transmit an oscillation or vibration to the human skin based on the position and the rotation angle of the at least one mechanical link. In this case, a movement such as a minute shaking or trembling applied to the object being controlled by remote may be transmitted to the skin of an operator as an oscillation or vibration.

The methods according to the above-described example embodiments may be recorded in non-transitory computer-readable media including program instructions to implement various operations embodied by a computer. The media may also include, alone or in combination with the program instructions, data files, data structures, and the like. The program instructions recorded on the media may be those specially designed and constructed for the purposes of the example embodiments, or they may be of the kind well-known and available to those having skill in the computer software arts. The media may also include, alone or in combination with the program instructions, data files, data structures, and the like. Examples of non-transitory computer-readable media include magnetic media such as hard disks, floppy disks, and magnetic tape; optical media such as CD ROM discs and DVDs; magneto-optical media such as optical discs; and hardware devices that are specially configured to store and perform program instructions, such as read-only memory (ROM), random access memory (RAM), flash memory, and the like.

Examples of program instructions include both machine code, such as produced by a compiler, and files containing higher level code that may be executed by the computer using an interpreter. The described hardware devices may be configured to act as one or more software modules in order to perform the operations of the above-described embodiments, or vice versa. Any one or more of the software modules described herein may be executed by a dedicated processor unique to that unit or by a processor common to one or more of the modules. The described methods may be executed on a general purpose computer or processor or may be executed on a particular machine such as the tactile feedback apparatus described herein.

Although example embodiments have been shown and described, it would be appreciated by those skilled in the art that changes may be made in these example embodiments without departing from the principles and spirit of the disclosure, the scope of which is defined in the claims and their equivalents.

What is claimed is:

1. A tactile feedback apparatus comprising:
a position measurement unit to measure a position of a mechanical link; and
a tactile feedback unit to transmit a tactile sensation to human skin based on at least one of a position of the mechanical link and a rotation angle of the mechanical link.

2. The tactile feedback apparatus of claim 1, wherein the tactile feedback unit is attached to at least one mechanical link included in the position measurement unit to transmit the tactile sensation.

3. The tactile feedback apparatus of claim 1, further comprising:
an operation control unit attached to the mechanical link and configured to perform a forceps operation.

4. The tactile feedback apparatus of claim 3, wherein the tactile feedback unit is attached to an outer surface of the forceps included in the operation control unit to transmit the tactile sensation.

5. The tactile feedback apparatus of claim 1, wherein the tactile feedback unit transmits the tactile sensation by moving at least one pin in at least one of a horizontal direction, a forward and backward direction, and a vertical direction, based on the position of the mechanical link and the rotation angle of the mechanical link.

6. The tactile feedback apparatus of claim 5, wherein the tactile feedback unit comprises three degrees of freedom for moving the at least one pin in at least one of the horizontal direction, the forward and backward direction, and the vertical direction, and
wherein the tactile feedback unit uses at least one of a piezo actuator, an ultrasonic actuator, and an electro active polymer to move the at least one pin.

7. The tactile feedback apparatus of claim 5, wherein the tactile feedback unit transmits at least one of a force, a movement, a texture, and a surface shape applied to an object to the human skin by moving the at least one pin.

8. The tactile feedback apparatus of claim 1, wherein the tactile feedback transmits the tactile sensation by adjusting a pneumatic pressure of a balloon according to the position of the mechanical link and the rotation angle of the mechanical link.

9. The tactile feedback apparatus of claim 1, wherein the tactile feedback unit transmits vibrations to the human skin based on the position of the mechanical link and the rotation angle of the mechanical link.

10. The tactile feedback apparatus of claim 1, wherein the tactile feedback unit transmits a force generated by a forceps operation of a remote controlled surgical robot to the human skin.

11. A tactile feedback method comprising:
measuring a position of a mechanical link; and
transmitting a tactile sensation to human skin based on at least one of the position of the mechanical link and a rotation angle of the mechanical link.

12. The tactile feedback method of claim 11, wherein the transmitting comprises transmitting the tactile sensation using a tactile feedback unit attached to at least one mechanical link included in a position measurement unit.

13. The tactile feedback method of claim 11, further comprising performing a forceps operation using an operation control unit attached to the mechanical link.

14. The tactile feedback method of claim 13, wherein the transmitting comprises transmitting the tactile sensation by a tactile feedback unit attached to an outer surface of the forceps included in the operation control unit.

15. The tactile feedback method of claim 11, wherein the transmitting comprises transmitting the tactile sensation by moving at least one pin in at least one of a horizontal direction, a forward and backward direction, and a vertical direction, based on the position of the mechanical link and the rotation angle of the mechanical link.

16. The tactile feedback method of claim 15, wherein the transmitting comprises moving the at least one pin in at least one of the horizontal direction, the forward and backward direction, and the vertical direction, using at least one of a piezo actuator, an ultrasonic actuator, or an electro active polymer to move the at least one pin.

17. The tactile feedback method of claim 15, wherein the transmitting comprises transmitting at least one of a force, a movement, a texture, and a surface shape applied to an object to the human skin by moving the at least one pin.

18. The tactile feedback method of claim 11, wherein the transmitting comprises transmitting the tactile sensation by adjusting a pneumatic pressure of a balloon according to the position of the mechanical link and the rotation angle of the mechanical link.

19. The tactile feedback method of claim 11, wherein the transmitting comprises transmitting vibrations to the human skin based on the position of the mechanical link and the rotation angle of the mechanical link.

20. The tactile feedback method of claim 11, wherein the transmitting comprises transmitting at least one of kinesthetic feedback and tactile feedback to the human skin based on the position of the mechanical link and the rotation angle of the mechanical link.

21. The tactile feedback method of claim 11, wherein the transmitting comprises transmitting a force generated by a forceps operation of a remote controlled surgical robot to the human skin.

22. A non-transitory computer readable recording medium storing a program to cause a computer to implement the method of claim 11.

23. A tactile feedback apparatus comprising:
a position measurement unit to measure a position of a mechanical link; and
a tactile feedback unit to transmit a tactile sensation by moving at least one pin based on a position of the mechanical link.

24. A system for performing remotely-controlled surgery, the system comprising:
a surgical robot comprising a surgical tool, the surgical robot to measure a force generated by the surgical tool; and
a tactile feedback apparatus to transmit a tactile sensation to a hand of a surgeon based on the force generated by the surgical tool of the surgical robot.

25. The tactile feedback apparatus of claim 24, wherein the tactile sensation transmitted by the tactile feedback unit comprises one or more of a force, a movement, a texture, and a surface shape, corresponding to the force generated by the surgical tool.
